(19) United States
(12) Patent Application Publication
(10) Pub. No.: US 2020/0001121 A1
(43) Pub. Date: Jan. 2, 2020

(54) FLEXIBLE CONTROL AND GUIDANCE OF MINIMALLY INVASIVE FOCUSED ULTRASOUND

(71) Applicant: THE JOHNS HOPKINS UNIVERSITY, Baltimore, MD (US)

(72) Inventors: Amir Manbachi, Baltimore, MD (US); Jeffrey Siewerdsen, Baltimore, MD (US); Nicholas Ellens, Baltimore, MD (US); Xiaoxuan Zheng, Baltimore, MD (US); Micah Belzberg, Baltimore, MD (US); Alan Cohen, Owings Mills, MD (US); Henry Brem, Ellicott City, MD (US)

(21) Appl. No.: 16/489,423

(22) PCT Filed: Feb. 28, 2018
(86) PCT No.: PCT/US2018/020159

§ 371 Date: Aug. 28, 2019

Related U.S. Application Data
(60) Provisional application No. 62/464,511, filed on Feb. 28, 2017.

(51) Int. Cl. A61N 7/02; A61B 8/08

(52) U.S. Cl. CPC A61N 7/02; A61N 2007/0004; A61B 8/0841

(57) ABSTRACT

An embodiment in accordance with the present invention provides a transducer design for minimally invasive focused ultrasound (MIFU). The present invention allows flexible control of a focused ultrasound wave using mechanical and electrical control. The transducer array is implemented on a flexible substrate that can be mechanically controlled through two or more physical configurations. As with conventional electronic “steering,” the transducer elements can be controlled electronically to provide adjustable focus of the ultrasound. The combination of mechanical and electronic control provides the device a very flexible method for delivering focused ultrasound. The invention also includes a design that allows integration of ultrasound and endoscopic image guidance. The ultrasound guidance includes anatomical visualization and functional imaging (e.g. blood flow and coagulation of vasculature). The ultrasound imaging transducer is used for thermometry within the region of interest for treatment. Endoscopic imaging allows for improved understanding of tip location in real-time.

### CROSS REFERENCE TO RELATED APPLICATION

This application claims the benefit of U.S. Provisional Patent Application No. 62/464,511 filed on Feb. 28, 2017, which is incorporated by reference, herein, in its entirety.

### FIELD OF THE INVENTION

The present invention relates generally to medical devices. More particularly, the present invention relates to a device and methods for flexible control and guidance of minimally invasive focused ultrasound.

### BACKGROUND OF THE INVENTION

Focused ultrasound (FUS) or therapeutic ultrasound has been a target of investigations as a method to treat lesions and tumors, in various organs including the brain. For example, the treatment of tremor by transcranial (intact skull) FUS thalamotomy is currently a well-studied application in humans.

Commonly, focused ultrasound treatment has been guided by magnetic resonance imaging. However, use of magnetic resonance guidance can be cost prohibitive and is only available in surgical centers equipped for such a procedure. As a result, focused ultrasound is not used as predominantly. In addition to that, transcranial ultrasound experiences a large amount of attenuation while passing through the skull. As a result, attenuation is a challenge that needs to overcome. For example, in one study an ultrasound beam was successfully focused through an intact cranium, the version of the device used at the time did not provide enough power to reach the temperature threshold for coagulative necrosis.

Therefore, finding a solution to increase the efficiency of the treatment, while trying to keep the ultrasound attenuation and cost as low as possible is important for a better outcome. This is where minimally invasive approach can provide a setting of minimal exposure (and hence infection) yet offering other benefits. it would be advantageous to provide a device and methods for flexible control and endoscopic guidance of minimally invasive focused ultrasound.

### SUMMARY OF THE INVENTION

The foregoing needs are met, to a great extent, by the present invention, wherein in one aspect a device for focused ultrasound includes a therapeutic ultrasound array. The therapeutic array is comprised of a number of array elements that are configured to move relative to one another. The device includes an ultrasound transducer configured for imaging of a treatment site. The device also includes a controller for actuating movement of the therapeutic ultrasound array and movement of one or more of the number of array elements relative to others of the number of array elements.

In accordance with an aspect of the present invention, the device includes mechanical movement of the therapeutic ultrasound array. The device can include electronic movement of the therapeutic ultrasound array. The device can also include computer control of the therapeutic ultrasound array. The therapeutic ultrasound array can be controlled via movement and time delay. A joystick is configured for a medical professional to control the therapeutic array. An endoscopic camera is configured for image guidance of the therapeutic ultrasound array.

In accordance with another aspect of the present invention, a device for focused ultrasound includes a therapeutic ultrasound transducer. The therapeutic ultrasound transducer is configured to produce an acoustic beam. A radius of curvature of the acoustic beam is varied using mechanical and electronic focusing, source frequency, and focusing location. The device includes an imaging ultrasound transducer, configured for imaging of a treatment site. Additionally, the device includes a delivery device for guiding the therapeutic ultrasound transducer and the imaging ultrasound transducer to the treatment site.

In accordance with another aspect of the present invention a delivery device takes the form of a flexible catheter. The therapeutic transducer is positioned in a forward facing position. The device can include robotic control of the delivery device. A non-transitory computer readable medium is programmed for control of the device and processing of image data transmitted from the device. The device is configured for power between 25-40 W. The device can have a shape memory. Additionally, the device can be MRI compatible.

In accordance with still another aspect of the present invention, a method for focused ultrasound includes generating an acoustic beam with a therapeutic ultrasound transducer. A radius of curvature of the acoustic beam is varied using mechanical and electronic focusing, source frequency, and focusing location. The method includes generating an image view of a region of interest with an imaging ultrasound transducer. The method also includes delivering the imaging ultrasound transducer and the therapeutic ultrasound transducer to the region of interest. The method includes using robotic control for delivering the imaging transducer and the therapeutic transducer to the region of interest. The method further includes delivering the device to a brain through one of a group consisting of Kocher's point, the nasal cavity, through an eyebrow incision, Frazier's point, Dandy's point, Keen's point, and Paine's point.

19. The method includes articulating elements of the therapeutic ultrasound array. The method includes delivering the imaging ultrasound transducer and the therapeutic ultrasound transducer using a flexible catheter. The method also includes using a non-transitory computer readable medium for delivering the imaging ultrasound transducer and the therapeutic ultrasound transducer. The method includes delivering the therapeutic ultrasound transducer for one of a group consisting of ablation of a tumor, drug delivery, and theranostic imaging and treatment.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A and 1B illustrate image view of a device and surgical method according to an embodiment of the present invention.

FIG. 2 illustrates image views of exemplary ventricular morphology.

FIGS. 3A-3C illustrate perspective views of a device according to an embodiment of the present invention.

FIGS. 4A-4C illustrate perspective views of the distal end of the device, according to an embodiment of the invention.

FIG. 5A illustrates a rear perspective view of the distal end of the device, and

FIG. 5B illustrates a side view of the distal end of the device, both according to an embodiment of the present invention.

FIGS. 6A-6E illustrate perspective views of the ultrasound transducer probe of the present invention in relation to a miniaturized endoscopic camera, according to embodiments of the present invention.

FIGS. 7A and 7B illustrate perspective views of a handle, according to an embodiment of the present invention.

FIGS. 8A and 8B illustrate perspective views of a forward viewing probe and array, according to an embodiment of the present invention.

FIG. 9 and FIG. 10 illustrate graphical views of acoustic beam profiles resulting from the therapeutic array elements, according to an embodiment of the present invention.

FIG. 11 illustrates graphical views of radial and lateral acoustic profiles of the beam patterns shown in FIGS. 9 and 10.

FIG. 12 illustrates graphical views of counter plots (−3 dB, −6 dB, and −12 dB) associated with the acoustic beam profiles resulting from the therapeutic array elements possessing curvatures relative to one another.

FIG. 13 illustrates graphical views of temperature map profiles resulting from the therapeutic array elements.

FIG. 14 illustrates a graphical view of a simulated amount of time for the heat deposition and hence temperature rise in white matter.

FIG. 15 illustrates a top down view of an acoustic beam profile, according to an embodiment of the present invention.

FIG. 16A illustrates a top down view of acoustic beam profiles at a thermal dose threshold of 240 minutes. FIG. 16B illustrates a graphical view of power versus lesion volume.

FIG. 17A illustrates focus position, according to an embodiment of the present invention. FIG. 17B illustrates acoustic beam profiles for one of the focus positions, and FIG. 17C illustrates a graphical view of lesion volume versus frequency.

FIG. 18A illustrates focus position, according to an embodiment of the present invention. FIG. 18B illustrates acoustic beam profiles for the focus positions, and FIG. 18C illustrates a graphical view of lesion volume versus power.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a novel transducer design for minimally invasive focused ultrasound (MIFU). The device of the present invention allows flexible control of a focused ultrasound wave using a combination of mechanical and electrical control. The transducer array is implemented on a flexible substrate that can be mechanically controlled through two or more physical configurations. As with conventional electronic “steering,” the driving phase and amplitude of the transducer elements can be controlled electronically to provide adjustable focus of the ultrasound. The combination of mechanical and electronic control provides the MIFU system a very flexible and fast, yet accurate method for delivering focused ultrasound.

In contrast to expensive MR-guided ultrasound treatment, the invention also includes a design that allows integration of ultrasound and endoscopic image guidance. The ultrasound guidance entails not only anatomical visualization, but also functional imaging (e.g. blood flow and coagulation of vasculature). The ultrasound imaging transducer is also employed as a means of collecting real-time thermometry and stiffness data within the region of interest for treatment. The endoscopic imaging allows for a better understanding of where the tip of the probe is located anatomically in real-time.

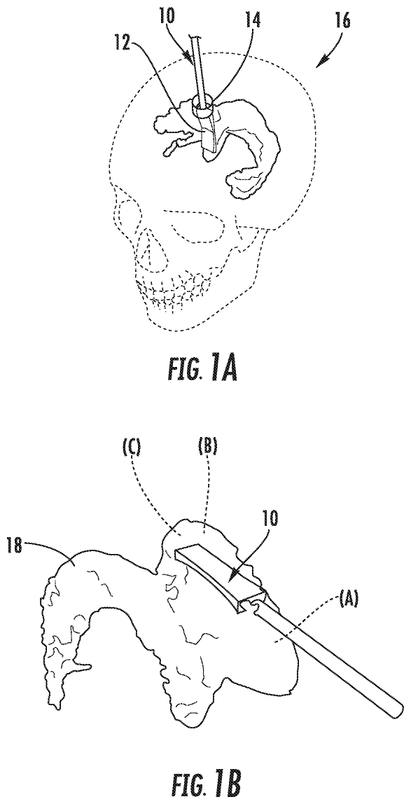

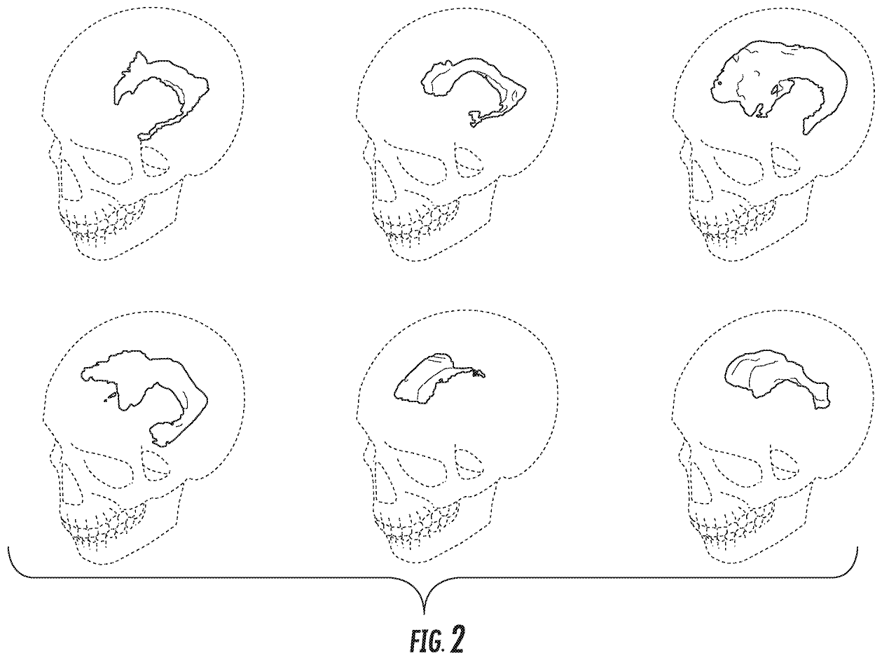

FIGS. 1A and 1B illustrate image view of a device and surgical method according to an embodiment of the present invention. As illustrated in FIG. 1A, a device 10 according to an embodiment of the present invention is used to apply focused ultrasound. The focused ultrasound applied by the device can directly treat the tumor, through thermal ablation/coagulation necrosis or mechanical cavitation. While ablation and cavitation are used as examples herein any treatment modality or other use for the device known to or conceivable to one of skill in the art is also included. Alternatively it can be used to indirectly treat the tumor (e.g. sensitizing tumors to radiotherapy via a hyperthermia-based mechanism, or opening the blood-brain barrier for drug delivery) 12. In the example illustrated in FIG. 1A, the tumor 12 is ablated from within the ventricles of the brain using real-time ultrasound guidance. The device 10 is equipped with this real time ultrasound guidance through transducers positioned on the device. The device of the present invention can be inserted through a burr hole 14 in the skull 16 of the patient, instead of subjecting the patient to a craniotomy. As will be described further herein, the device 10 can be steered and the shape can be changed in order to ablate the tumor(s) from within the ventricle. While the device is illustrated herein with respect to use in the brain, it is not to be considered limiting, and it is to be understood that the device can be used to apply focused ultrasound in any other anatomical region where it may be therapeutic. The burr hole 14 could be placed in a number of locations including, but not limited to, Kocher's point, the nasal cavity, through an eyebrow incision, Frazier's point, Dandy's point, Keen's point, and Paine's point. This list of access points for the burr hole should not be considered limiting, and any point of entry known to or conceivable by one of skill in the art could also be used. FIG. 1B illustrates a device 10 inserted into a lateral ventricle 18 of the brain. As illustrated in FIG. 1B, several access points are available including Kocher's point (with a burr hole diameter of 1-1.5 cm) (A), Keen's point (B), and Dandy's point (C).

FIG. 2 illustrates image views of exemplary ventricular morphology. As illustrated in the 6 examples, the shape of the ventricles can vary widely from patient to patient. This variation in ventricular morphology illustrates the importance of being able to ablate/cavitate and visualize the lesions from within the ventricle. Therefore, the device of the present invention is well suited providing focused ultrasound ablation/cavitation of tumors within or near the ventricle. The variability of the patient-specific anatomy demonstrated in FIG. 2 also hints to the fact that the tip of the device may not be able to bend/curve in any manner desirable (anatomy/dimensions limitations) That is why electronic focusing, on top of the mechanical flexibility, allows for better control over the focusing of the ultrasound beam in 3D space within the desired organ.

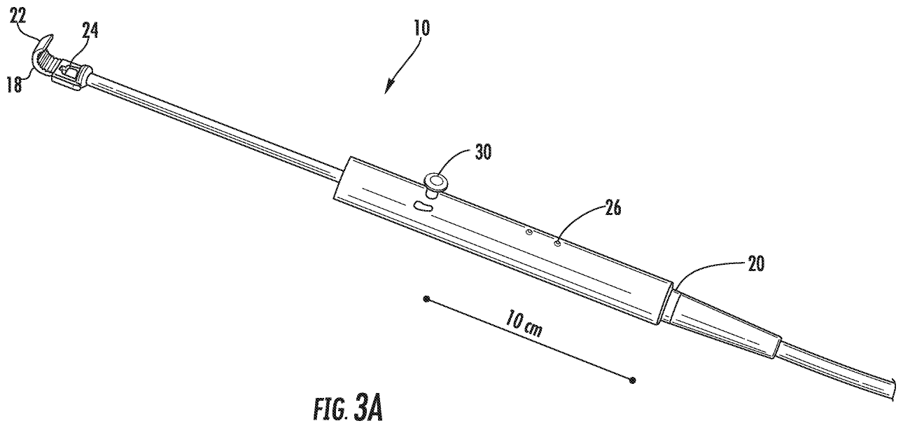

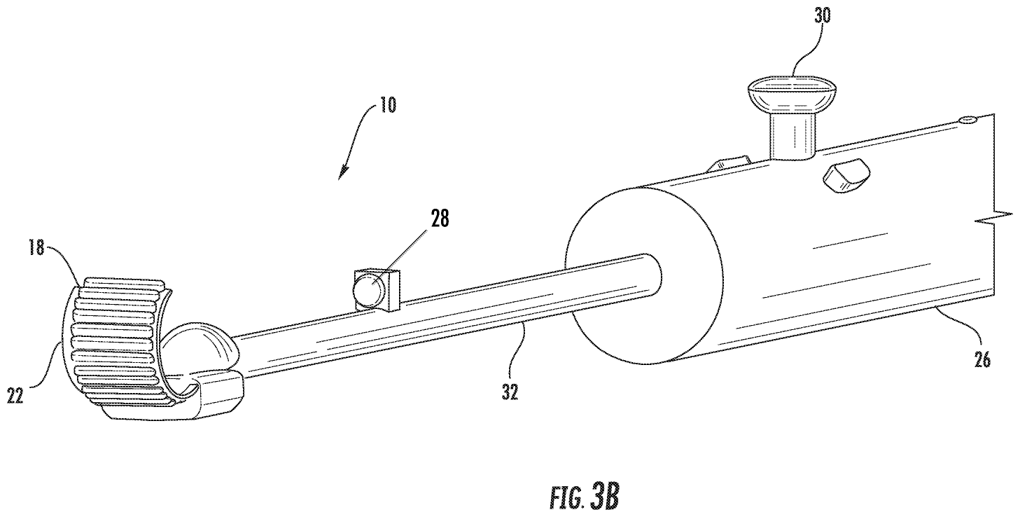

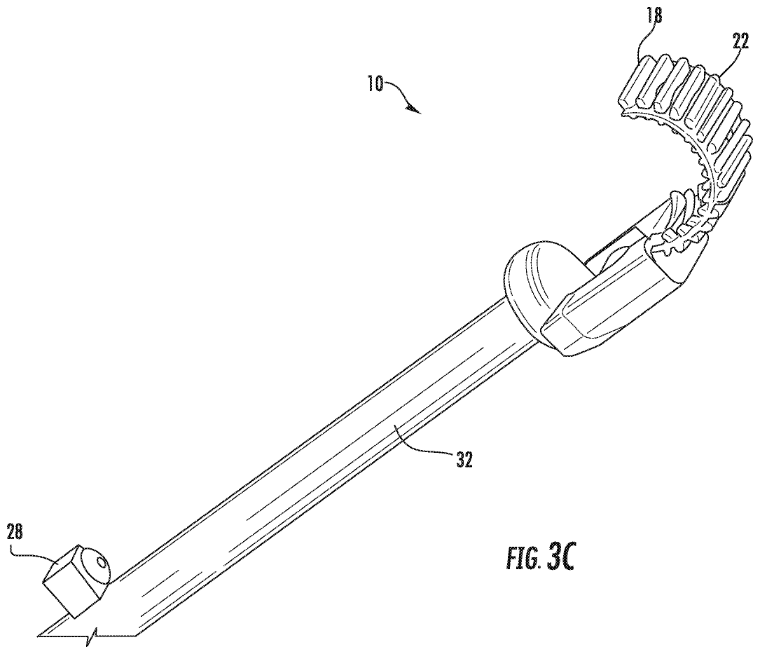

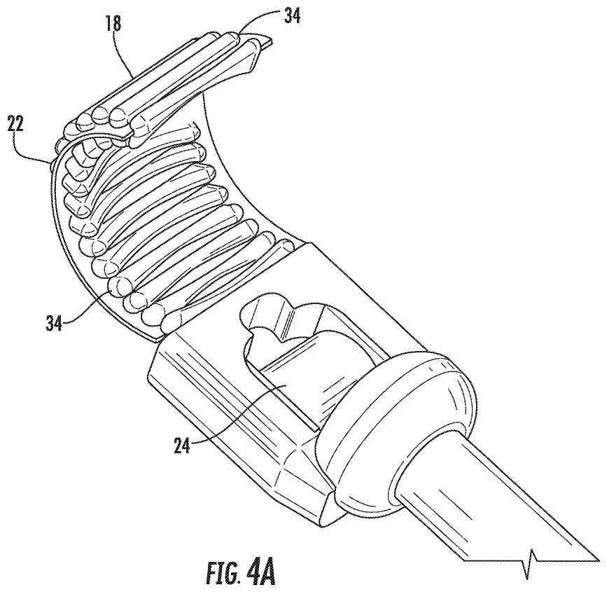

FIGS. 3A-3C illustrate perspective views of a device according to an embodiment of the present invention. As illustrated in FIGS. 3A-3C, the device 10 includes a distal end 18 and a proximal end 20. A flexible array 22 for treatment and an ultrasound probe 24 for imaging is positioned at the distal end 18 of the device 10 and a handle 26 is positioned at the proximal end of the device 10. Between the flexible array 22 and the handle 26 is positioned an endoscopic camera 28 for further visualization of the distal end 18 of the device 10. The handle 26 includes a joy stick 30 to provide control over the curvature and shape of the flexible array 22. The joy stick 30 can provide mechanical and/or electrical control over the shape and curvature of the flexible array 22. The endoscopic camera 28 is positioned on an elongate body 32 of the device 10. The endoscopic camera 28 can be miniaturized for visualizing the distal end 18 of the device 10, which contains the real-time ultrasound imaging and thermometry transducer, and the flexible array 22 of therapeutic ultrasound elements. The real-time ultrasound imaging transducer and the thermometry transducer can take the form of a single transducer, multiple transducers, or separate transducers dedicated to each function.

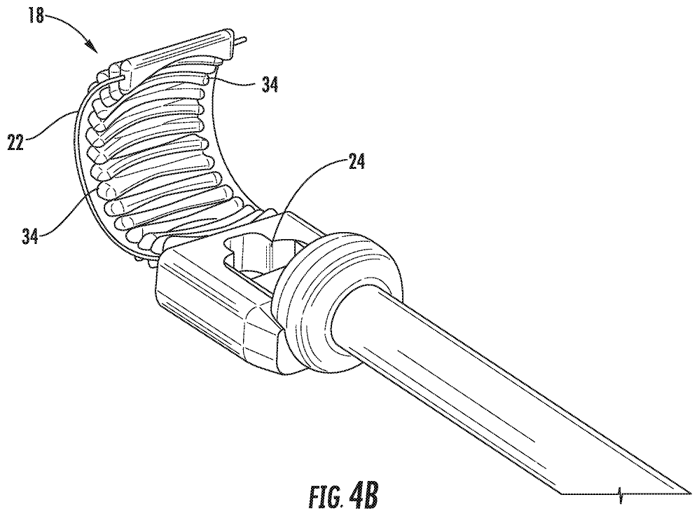

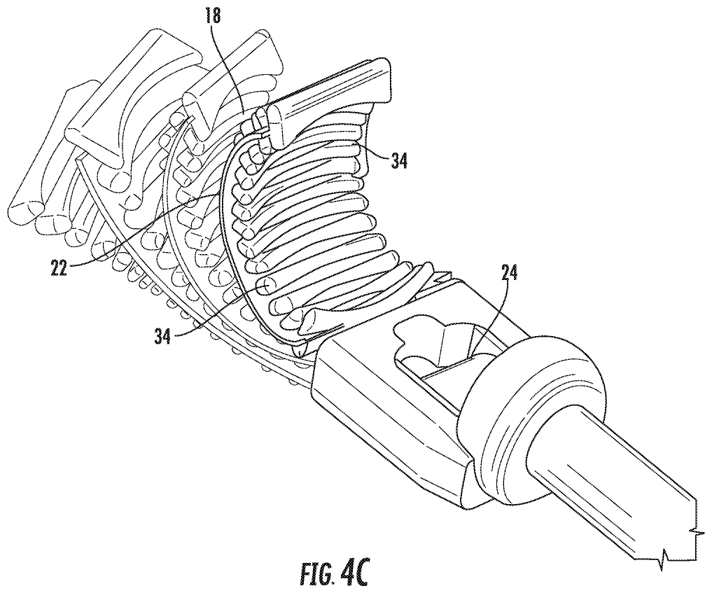

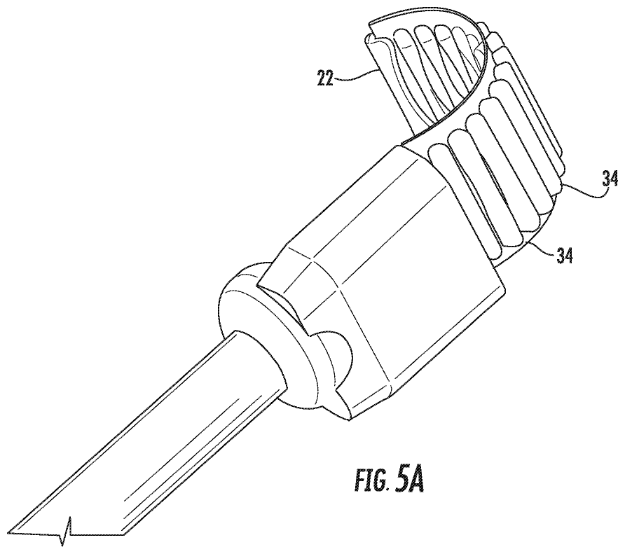

FIGS. 4A-4C illustrate perspective views of the distal end of the device, according to an embodiment of the invention. As illustrated in FIGS. 4A-4C the distal end 18 of the device 10 includes both the flexible array 22 and the ultrasound probe 24. The flexible array 22 is configured for treatment, while the ultrasound probe 24 includes both the imaging and thermometry transducing elements. The flexible array can have mechanical flexibility and the individual array elements 34 can possess various curvatures relative to one another. The individual array elements 34 an pivot with respect to one another, to provide the flexible array with a range of movement and flexible three-dimensional focusing. Each of the individual array elements can also be regulated independently by a driver console, allowing for electronic steering and faster, enhanced treatment. Electronic steering can be actuated by the medical professional alone, or in conjunction with surgical robotics and/or computer control.

A position of the tip of the device of the present invention (both the therapeutic array elements and the imaging transducers) relative to the anatomy is visualized through a miniaturized endoscopic camera that allows for image-guided navigation of the probe. The therapeutic array elements are mounted on a flexible substrate that allows mechanical flexibility, and hence various curvatures being possessed by the array. The therapeutic array elements are mounted on a flexible substrate that allows remembrance of mechanical configurations via the employment of memory metals and mechanical and/or electrical control, via the use of a jig/joy stick mounted on the handle.

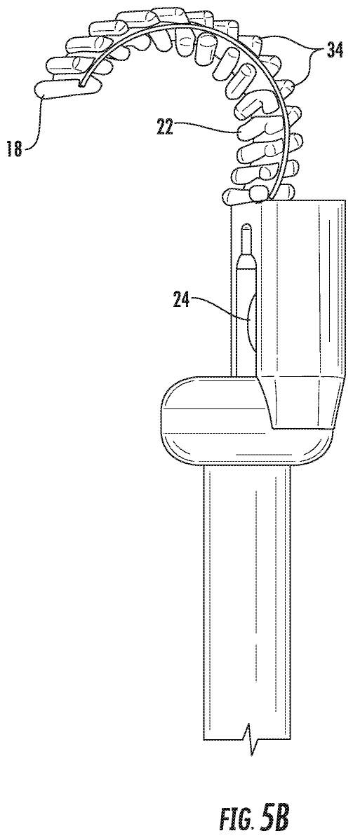

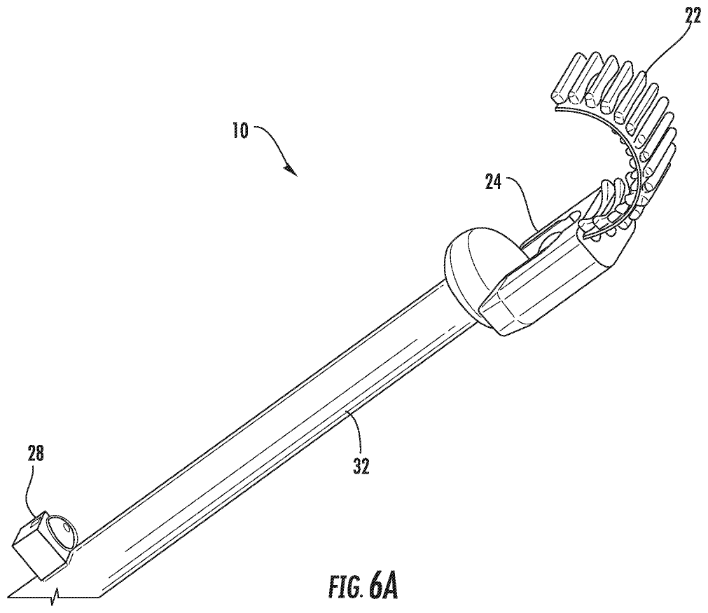

FIG. 5A illustrates a rear perspective view of the distal end of the device, and FIG. 5B illustrates a side view of the distal end of the device, both according to an embodiment of the present invention. FIGS. 5A and 5B illustrate the position of the flexible array 22 relative to the ultrasound transducer probe 24. The individual array elements 34 are also illustrated in further detail in FIGS. 5A and 5B. In some embodiments the flexible array 22 can be fixed into a rigid transducer.

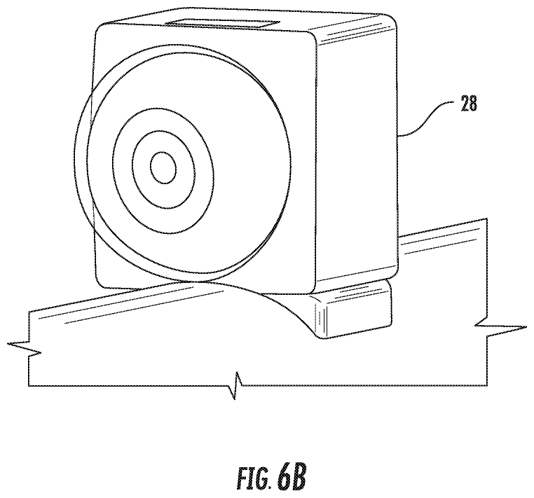

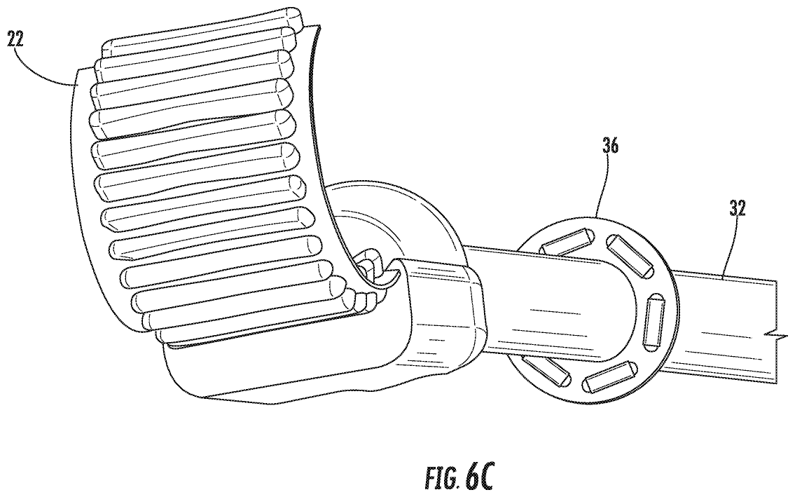

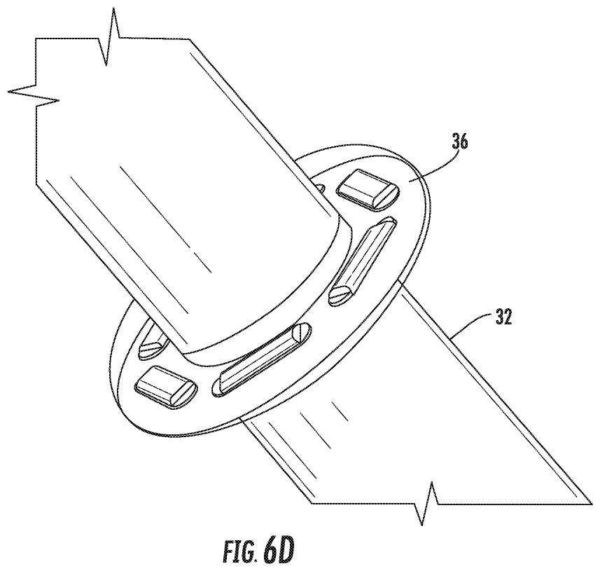

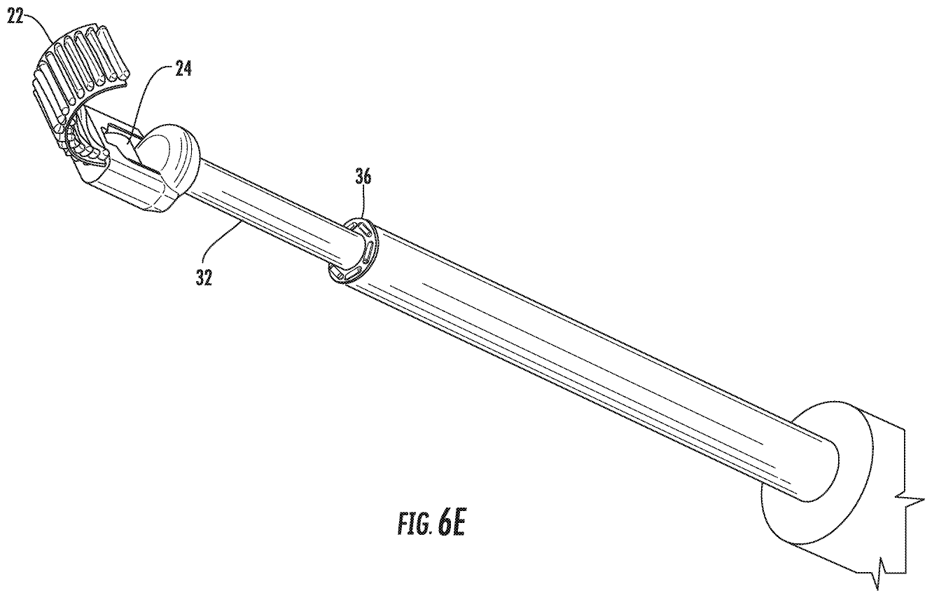

FIGS. 6A-6E illustrate perspective views of the ultrasound transducer probe of the present invention in relation to a miniaturized endoscopic camera, according to embodiments of the present invention. FIG. 6A illustrates the endoscopic camera 28 being positioned on the elongate body 32 of the device 10, proximal to the ultrasound transducer 24. FIG. 6B illustrates a perspective view of the endoscopic camera 28 in greater detail. FIGS. 6C-6E illustrate an endoscopic array 36 being positioned on the elongate body 32 of the device 10. The endoscopic array 36 is again positioned proximal to the ultrasound transducer 24.

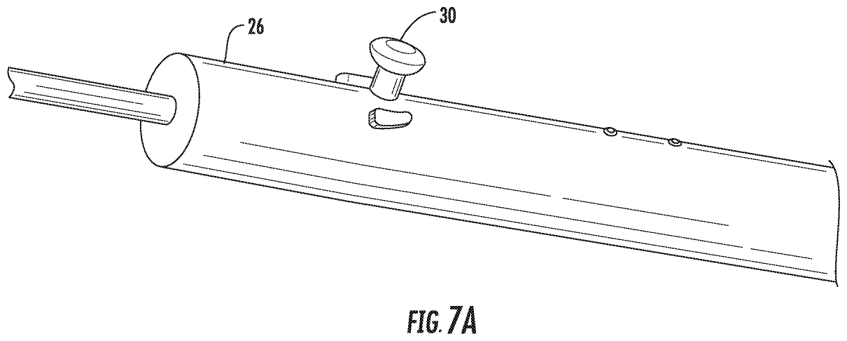

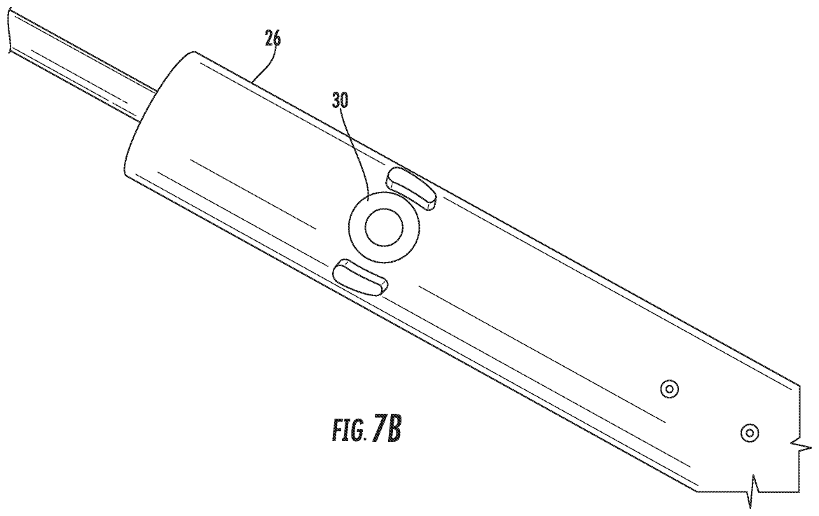

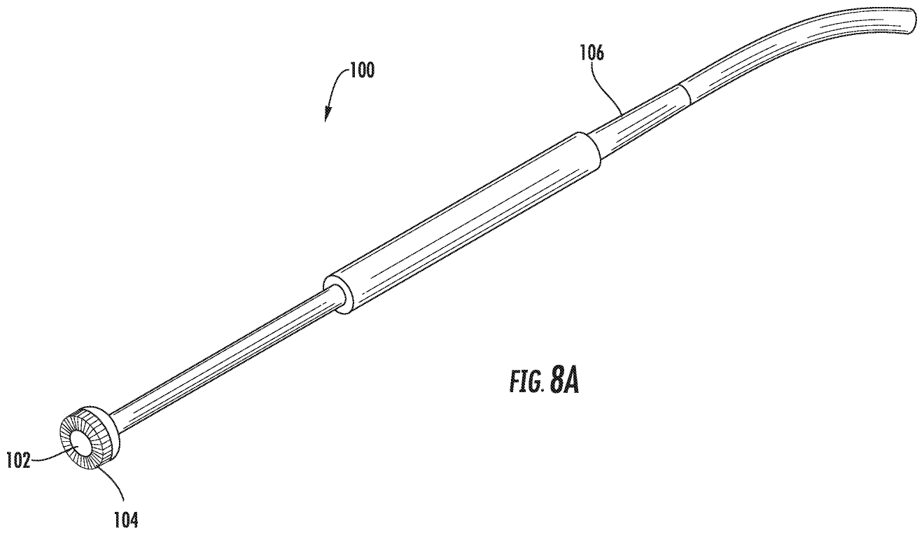

FIGS. 7A and 7B illustrate perspective views of a handle, according to an embodiment of the present invention. The handle 26 includes the joystick 30 for controlling movement of the array (not pictured). The joystick 30 can provide mechanical and/or electronic control of the array. The joystick 30 can also be computer enabled for further guidance and steering of the array.

The joystick mounted on the handle allows for the therapeutic array elements to be curved inwards or outwards relative to the axis of the handle. As a result of this mechanical flexibility and control, various curvatures can be possessed by the array. The joystick mounted on the handle also allows for the therapeutic array elements to be curved to the sides (e.g. left of right) relative to the axis of the handle. As a result of this mechanical flexibility and control, various curvatures can be possessed by the array in a second degree of freedom. In some embodiments of this invention, the therapeutic array elements can be controlled by a software installed on the driver of the probe to utilize each of the array elements independently. The above-mentioned driver, can be connected to the probe through a cable, wirelessly, via Bluetooth, RFID, or any other communication modality known to or conceivable to one of skill in the art.

In some embodiments of this invention, the above-mentioned driver, can be utilized to display the real-time ultrasound images of the treatment region, the real-time thermometry images of the treatment region, and the real-time endoscopic images of the anatomical region. The therapeutic array elements can be controlled by a software installed on the driver of the probe to fire acoustic energies in delayed timing fashion relative to one another or to fire acoustic energies simultaneous, though in various signal phase relative to one another.

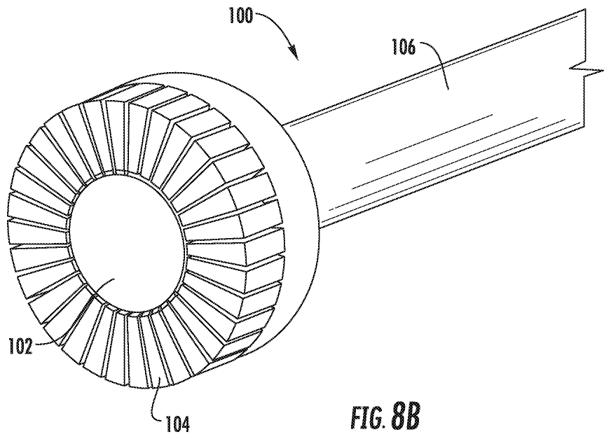

FIGS. 8A and 8B illustrate perspective views of a forward viewing probe and array, according to an embodiment of the present invention. FIGS. 8A and 8B illustrate a device 100 with a forward facing therapeutic element 102. The forward facing therapeutic element 102 can take the form of one element or a number of elements combined to provide the therapeutic acoustic wave. The forward facing therapeutic element is circled by an array of imaging elements 104. The array of imaging elements can fill the circle or imaging elements can be placed at a predetermined spacing around the therapeutic element 102. The therapeutic element 102 and the array of imaging elements 104 are positioned at a distal end of a handle 106. The handle 106 can be rigid or flexible and in some embodiments can take the form of a catheter for delivering the therapeutic element 102 and the array of imaging elements 104 to the desired location.

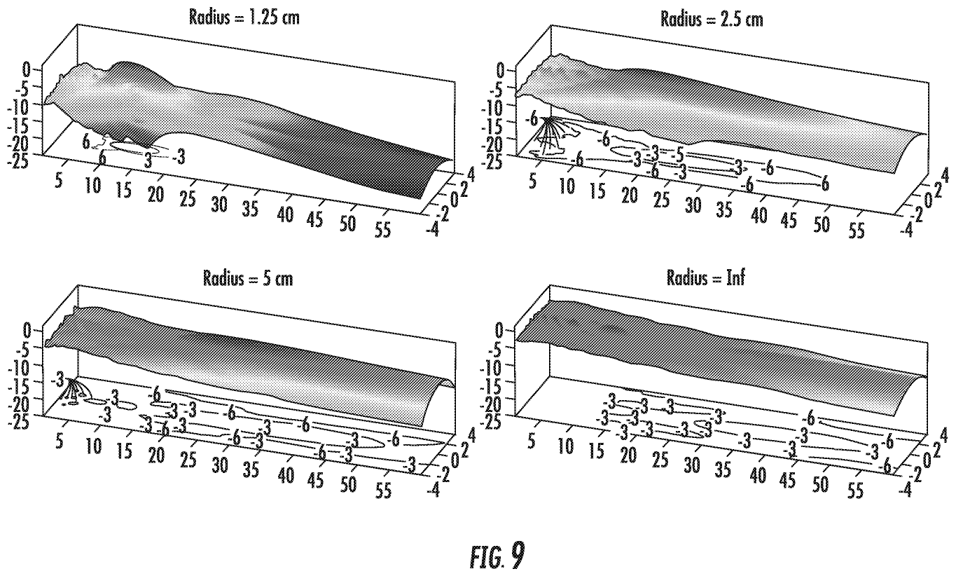

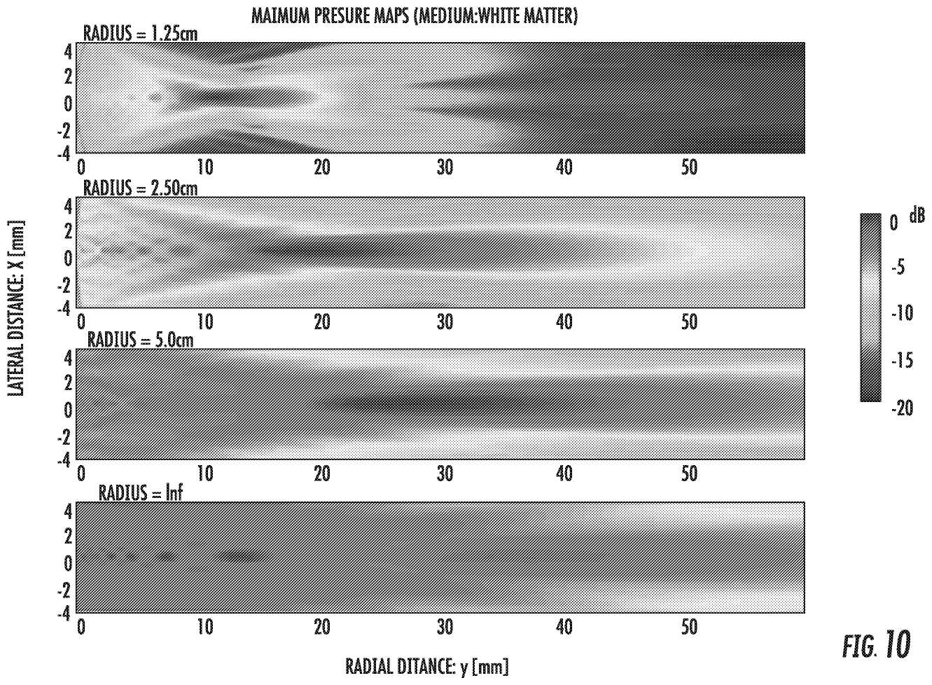

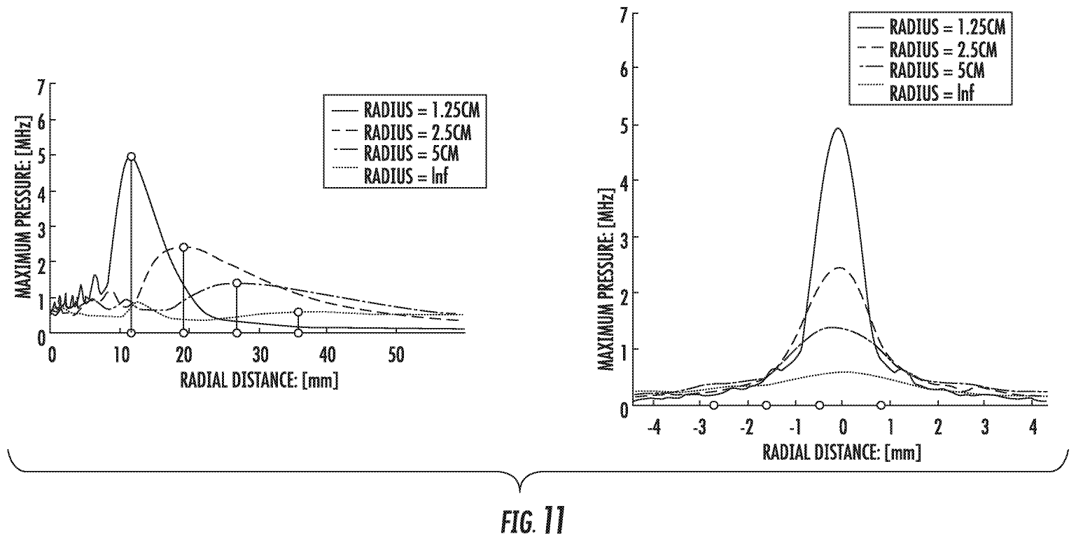

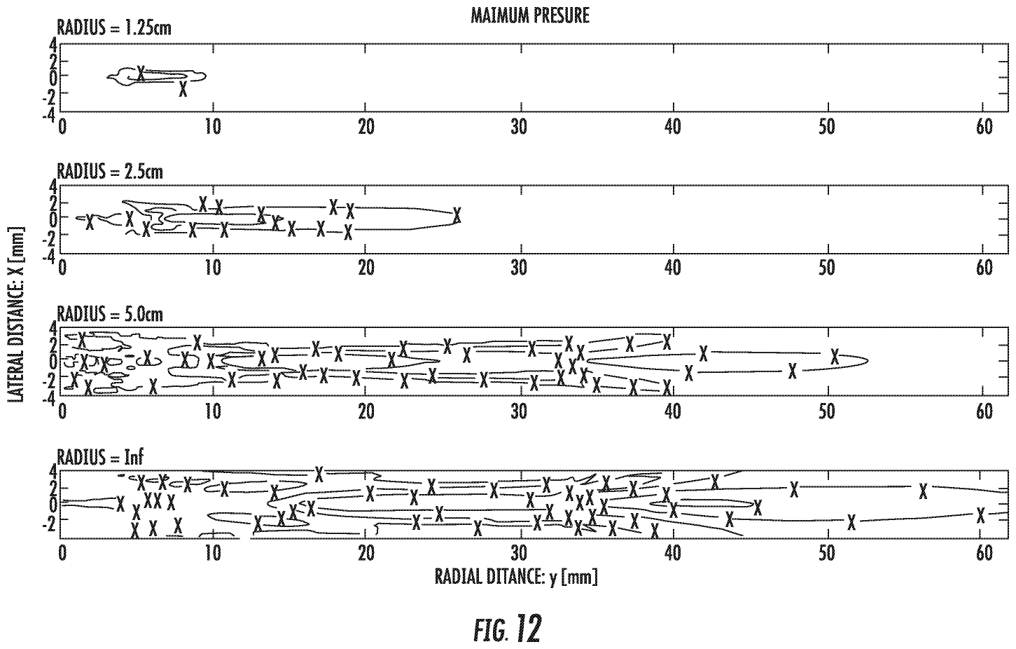

FIG. 9 and FIG. 10 illustrate graphical views of acoustic beam profiles resulting from the therapeutic array elements, according to an embodiment of the present invention. The acoustic beam profiles are associated with varying curvatures of the therapeutic array elements, relative to one another. Shown in different lines are the few exemplary radii of curvatures. In FIGS. 9 and 10 the medium is assumed to be white matter. FIG. 9 illustrates a perspective view of the acoustic beam profiles, and FIG. 10 illustrates a top down view of the acoustic beam profiles. FIG. 11 illustrates graphical views of radial and lateral acoustic profiles of the beam patterns shown in FIGS. 9 and 10. FIG. 12 illustrates graphical views of counter plots (−3 dB, −6 dB, and −12 dB) associated with the acoustic beam profiles resulting from the therapeutic array elements possessing curvatures relative to one another. Shown in different lines are the few exemplary radii of curvatures, demonstrating the capability of the array to focus the beams anywhere from 1 to 7 cm. Here the medium is assumed to be water, resembling cerebrospinal fluid (CSF).

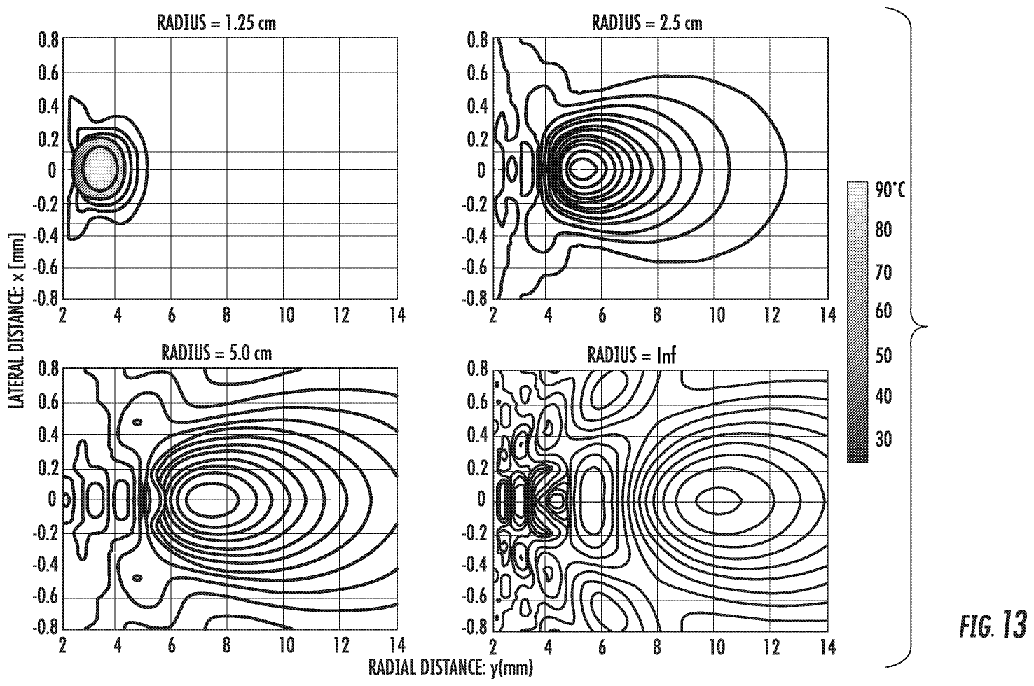

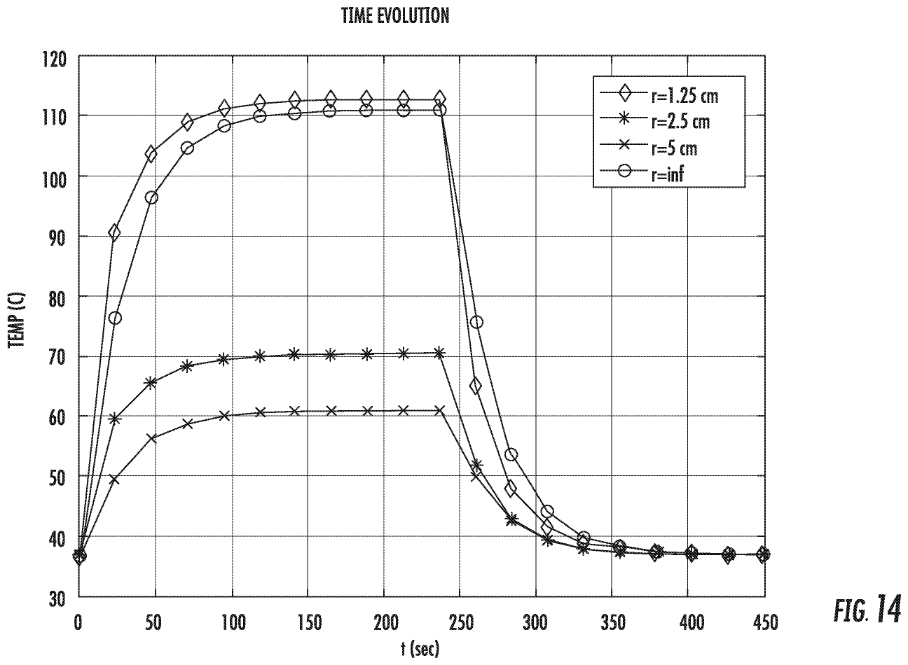

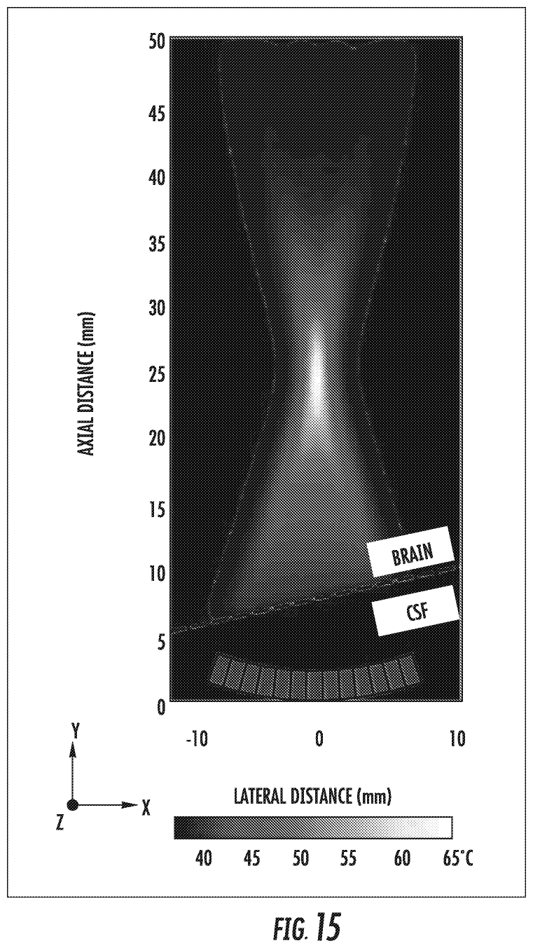

FIG. 13 illustrates graphical views of heat map profiles resulting from the therapeutic array elements. The therapeutic array elements possess varying curvatures relative to one another. FIG. 13 shows the few exemplary radii of curvatures. Here the medium is assumed to be water, resembling CSF. FIG. 14 illustrates a graphical view of a simulated amount of time for the heat deposition and hence temperature rise in white matter.

FIG. 15 illustrates a top down view of an acoustic beam profile, according to an embodiment of the present invention. The acoustic beam profile shows the lateral distance of the beam plotted against the axial distance of the beam with a heat map, based on the key at the bottom of the graph. Three major variables were identified that change the radius of curvature for the acoustic beam profile: mechanical and electronic focusing, source frequency, and focusing location. Table 1, below shows the acoustic parameters for a simulation study done to investigate the impact of ablation on the target tissue.

TABLE 1
Acoustic Parameters

| Medium | | |
|---|---|---|
| Brain | 1030 | 1545 |
| CSF | 995 | 1510 |
| 5.30 | 3640 | 0.54 |
| 2.65 ×. 10⁻² | 4200 | 0.62 |

Target Temperature: 65° C.
Sonication Time: 5 seconds
Cooling Period: 90 seconds

The acoustic pressure profile is calculated with the Westervelt Equation p(x, y, z). Next, the temperature distribution is calculated with the Pennes' Bioheat Transfer Function:

$${ρ}_{t} {C}_{t} \frac{\partial T}{\partial t} = {k}_{t} {∇}^{2} T - {ρ}_{b} {C}_{b} w ( T - {T}_{0} ) + Q$$

The dosage calculation is then completed with the Thermal Dose Function:

T₄₃(x, y, z)=∫R^{43-T(x,y,z,t)}dt

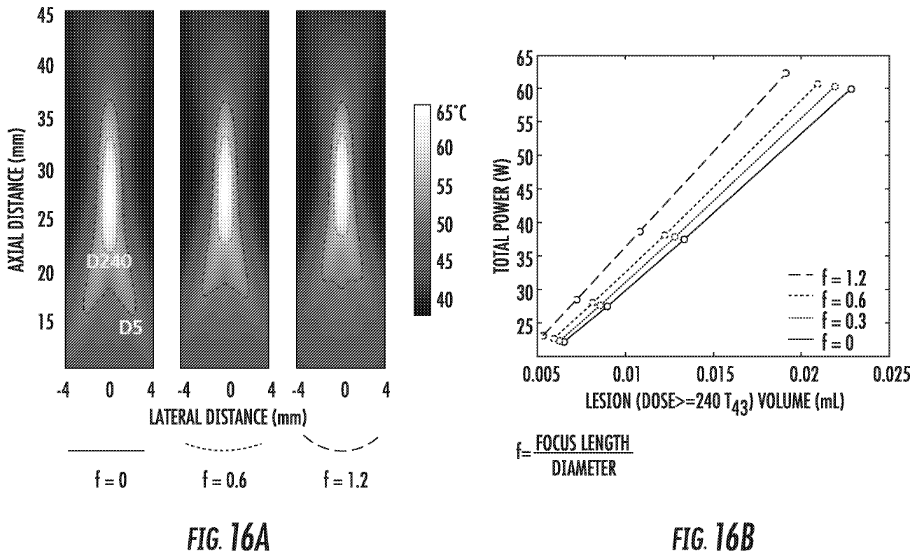

FIG. 16A illustrates a top down view of acoustic beam profiles at a thermal dose threshold of 240 minutes. f varies for each plot, where f is the focus length divided by the diameter of the beam. FIG. 16B illustrates a graphical view of power versus lesion volume. A thermal dose threshold of 240 min (in equivalent time at 43C) would produce damage and necrosis of brain tissue, and a thermal dose threshold greater than 5 min and less than 240 min is considered the transition region, in which the tissue may or may not be damaged. A focus position is fixed at 30 mm. An acoustic pressure profile is scaled to reach 65 C in 5 seconds of sonication time. This scale factor is proportional to the acoustic power, and is used to obtain a plot of power versus lesion volume. A tighter lesion results when both mechanical and electronic focusing is used, but more power is required.

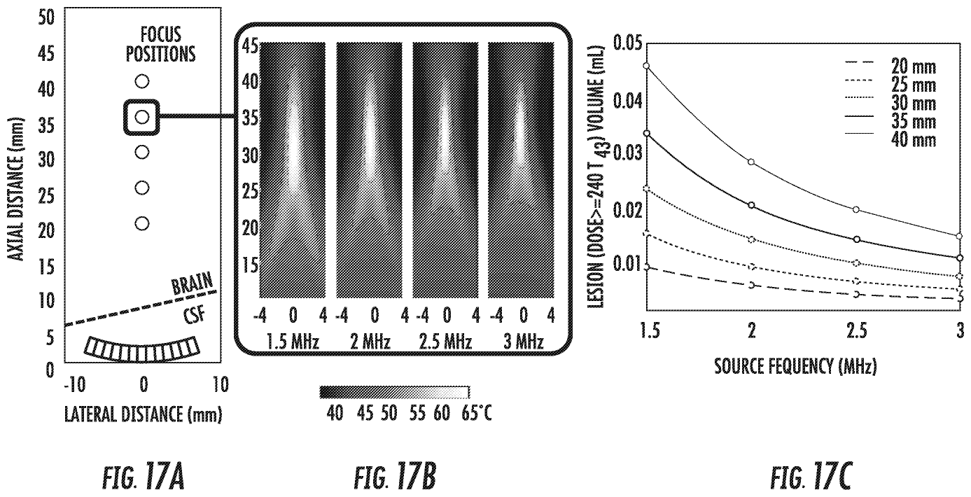

FIG. 17A illustrates focus position, according to an embodiment of the present invention. FIG. 17B illustrates acoustic beam profiles for one of the focus positions, and FIG. 17C illustrates a graphical view of lesion volume versus frequency. Only electronic steering was used to focus the beam. High frequency is favorable for creating precise and small lesioning in further focus positions.

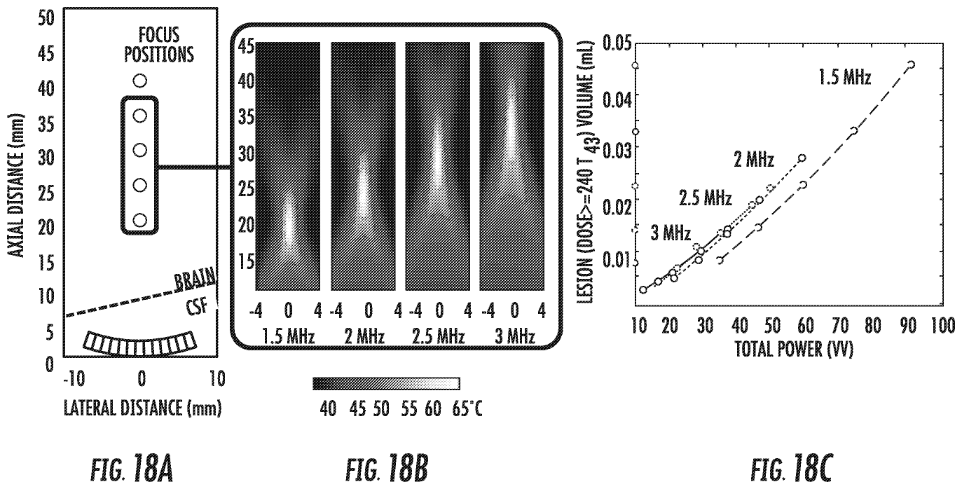

FIG. 18A illustrates focus position, according to an embodiment of the present invention. FIG. 18B illustrates acoustic beam profiles for the focus positions, and FIG. 18C illustrates a graphical view of lesion volume versus power. Tuning frequency allows for the creation of a lesion of approximately the same size at different focus locations. A lesion with a depth of 1 cm and a width of 2 mm can be generated using a power of 25-40 W.

The present invention is directed to a device with a novel transducer design that allows treatment of lesions (e.g. cysts, or tumors) or facilitating drug delivery through opening the blood-brain barrier using intracranial ultrasound. The transducer includes a plurality of therapeutic transducer elements arranged in a flexible, steerable configuration at the tip of the device. The transducer array includes transducers for therapy, as well as imaging transducers.

In some embodiments, the imaging and therapeutic transducers can be interchangeable. In other embodiments, the device can take the form of a flexible catheter with an imaging and therapeutic component. The treatment region is imaged in real-time by an imaging ultrasound transducer, such as a convex probe with a large field of view to cover surrounding tissues. The design is unique and allows minimally invasive insertion of the device and then the mechanical flexibility of the therapeutic tip enables flexibility over the focusing of the beam in 3D space and even if the anatomy/dimensions were limiting the tip from free movement and bending, the electronic focusing can take over and help with the objective of focusing the ultrasound beam wherever necessary. This device is useful for any cavity that is hard to reach and a small minimally invasive approach would be beneficial. Any imaging ultrasound transducer known to or conceivable to one of skill in the art can be used for imaging the treatment region. In some embodiments of this invention, the imaging ultrasound transducer can be a one-dimensional or two-dimensional array, enabling a better understanding of the extent of the ablation and cavitation lesions. The treatment region is monitored in real-time for temperature changes by ultrasound thermometry, using the same imaging transducer described above. The device can have shape-memory properties and can also be formed from MRI-safe materials.

The above-described features (i.e. curvature flexibility, mechanical memory, and control of the therapeutic array elements) enable robust control on the three-dimensional positioning of the focal spots associated with the acoustic profiles resulting from the curved array, of the energy deposition associated with the heat maps resulting from the curved array, and of the temperature peaks/mechanical cavitations resulting from the curved array and on the three dimensional resolution of the energy deposition associated with the heat maps resulting from the curved array. The above-described mechanical control of the array curvature is achieved through the use of a flexible substrate with spring-like mechanical properties.

In some embodiments of this invention, the therapeutic array elements can be curved mechanically and also controlled electronically or robotically. The above-described features (i.e. curvature flexibility, mechanical memory, configuration control of the therapeutic array elements and electronic regulator over independent array elements) enables fine tuning the resolution and location of the focal spot. In some embodiments of this invention, the focal points of the heat maps possess circular and oval shapes, resembling rice grain contour. In some embodiments of this invention, the three-dimensional ablation of the brain lesions, cysts and tumors can be accomplished through ablation/cavitation of the lesion by ablating/cavitating a number of “rice grain” oval segments, one at a time. The intersection of such “rice grain” oval segments coincides with the center of the therapeutic array elements. Faster ablation/cavitation of three-dimensional structures can be achieved through the use of electronic regulation of the array elements, and hence electronic steering of the sonic beams. The above-mentioned point can eliminate the need for mechanical rotation, unless deemed necessary by the operator/clinician or when necessary due to anatomical limitations discouraging the mechanical bending of the therapeutic array. In summary, the present invention is directed to a system and method for compacting a plurality of therapeutic transducer elements arranged in a flexible configuration, an ultrasound imaging probe and an endoscopic camera in a fashion that minimizes the dimension of the minimally invasive burr hole and allows for configuration variation within the hard-to-reach cavities (e.g. brain ventricles), enabling flexibility in the treatment efficacy. While the device is described with respect to treatment of lesions in the brain, the device of the present invention can be used in a number of applications and treatments including a focused transducer for ablation of tumors, drug delivery and theranostic imaging and treatment.

Control of the device and display of visual images or data related to the device and procedure of the present invention can be carried out using a computer, non-transitory computer readable medium, or alternatively a computing device or non-transitory computer readable medium incorporated into the robotic device or the imaging device. Fundamentally (from a physics standpoint) to maximize the heat deposition, spherical bowl shaped transducers are used. Unlike other HIFU (High-Intensity Focused Ultrasound) devices that require a cooling mechanism, the advantage of our device sitting in a fluid-filled cavity (such as brain ventricles) is that it does not require a cooling mechanism.

The device of the present invention can be used to look into the field to see how much tumor is left, especially in areas that are difficult to expose and visualize. This device can in real time provide better feedback during the resection as it can be used in the resection cavity as opposed to the brain surface. The resection could be done with real time imaging feedback.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The computing device can also take the form of an operating console computer. The operating console is a non-generic computer specifically designed by the manufacturer. It is not a standard business or personal computer that can be purchased at a local store. Additionally, the console computer can carry out communications with the scanner through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for focused ultrasound comprising:
a therapeutic ultrasound array, wherein the therapeutic ultrasound array is comprised of a number of ultrasound array elements that are configured to move relative to one another;
an ultrasound transducer configured for imaging of a treatment site; and
a controller for actuating movement of the therapeutic ultrasound array and movement of one or more of the number of ultrasound array elements relative to others of the number of ultrasound array elements.

2. The device of claim 1 further comprising mechanical movement of the therapeutic ultrasound array.

3. The device of claim 1 further comprising electronic movement of the therapeutic ultrasound array.

4. The device of claim 1 further comprising computer control of the therapeutic ultrasound array.

5. The device of claim 1 wherein the therapeutic ultrasound array can be controlled via movement and time delay.

6. The device of claim 1 further comprising a joystick configured for a medical professional to control the therapeutic ultrasound array.

7. The device of claim 1 further comprising an endoscopic camera configured for image guidance of the therapeutic ultrasound array.

8. A device for focused ultrasound comprising:
a therapeutic ultrasound transducer, wherein the therapeutic ultrasound transducer is configured to produce an acoustic beam, and wherein a radius of curvature of the acoustic beam is varied using mechanical and electronic focusing, source frequency, and focusing location;
an imaging ultrasound transducer, configured for imaging of a treatment site; and
a delivery device for guiding the therapeutic ultrasound transducer and the imaging ultrasound transducer to the treatment site.

9. The device of claim 8 further comprising the delivery device taking the form of a flexible catheter.

10. The device of claim 8 further comprising the therapeutic ultrasound transducer being positioned in a forward facing position.

11. The device of claim 8 further comprising robotic control of the delivery device.

12. The device of claim 8 further comprising a non-transitory computer readable medium programmed for control of the device and processing of image data transmitted from the device.

13. The device of claim 8 wherein the device is configured for power between 25-40 W.

14. The device of claim 8 further comprising the device having a shape memory.

15. The device of claim 8 further comprising the device being MRI compatible.

16. A method for focused ultrasound comprising:
generating an acoustic beam with a therapeutic ultrasound transducer, wherein a radius of curvature of the acoustic beam is varied using mechanical and electronic focusing, source frequency, and focusing location;
generating an image view of a region of interest with an imaging ultrasound transducer; and
delivering the imaging ultrasound transducer and the therapeutic ultrasound transducer to the region of interest.

17. The method of claim 16 further comprising using robotic control for delivering the imaging ultrasound transducer and the therapeutic ultrasound transducer to the region of interest.

18. The method of claim 16 further comprising delivering the device to a brain through one of a group consisting of Kocher's point, a nasal cavity, through an eyebrow incision, Frazier's point, Dandy's point, Keen's point, and Paine's point.

19. The method of claim 16 further comprising articulating elements of the therapeutic ultrasound array.

20. The method of claim 16 further comprising delivering the imaging ultrasound transducer and the therapeutic ultrasound transducer using a flexible catheter.

21. The method of claim 16 further comprising using a non-transitory computer readable medium for delivering the imaging ultrasound transducer and the therapeutic ultrasound transducer.

22. The method of claim 16 further comprising delivering the therapeutic ultrasound transducer for one of a group consisting of ablation of a tumor, drug delivery, and theranostic imaging and treatment.
